(19) United States
(12) Patent Application Publication
(10) Pub. No.: US 2005/0065541 A1
(43) Pub. Date: Mar. 24, 2005

(54) Guarded surgical scalpel with blade stripper lock to prevent accidental or inadvertent ejection of the blade

(75) Inventors: Michael R. Abidin, Alexandria, VA (US); Steven P. Lehmbeck, Baltimore, MD (US)

Correspondence Address: ARMSTRONG, WESTERMAN & HATTORI, LLP;Intellectual Property Law Offices, Suite 220, 502 Washington Avenue, Towson, MD, 21204, US

(73) Assignee: ALB ASSOCIATES, LLC

(21) Appl. No.: 10/670,428

(22) Filed: Sep. 24, 2003

(51) Int. Cl. A61B 17/32

(52) U.S. Cl. 606/167

(57) ABSTRACT

A guarded surgical scalpel has a manually-releasable lock on its blade stripper to prevent inadvertent or accidental ejection of the blade off the scalpel.

### FIELD OF THE INVENTION

The present invention relates to a guarded surgical scalpel equipped with a blade stripper, and more particularly, to a guarded surgical scalpel having a blade stripper provided with a lock to prevent the inadvertent or accidental actuation of the blade stripper during use or handling of the scalpel.

### BACKGROUND OF THE INVENTION

In our previous patents -

U.S. Pat. No. 5,250,063

U.S. Pat. No. 5,275,606

U.S. Pat. No. 5,411,512

U.S. Pat. No. 5,569,281

we disclosed and claimed a guarded surgical scalpel to prevent accidental or inadvertent cuts during use or handling of the scalpel in an operating procedure.

In our additional patents -

U.S. Pat. No. 5,496,340

U.S. Pat. No. 5,662,669

we disclosed and claimed a guarded surgical scalpel having a blade stripper for ejecting a used blade from the scalpel without physically touching the used blade.

The disclosures of our earlier patents (listed above) are incorporated in their entirety by reference herein.

The present invention constitutes a further improvement over these earlier patents.

### SUMMARY OF THE INVENTION

The present invention has particular utility in a guarded surgical scalpel, wherein a blade is mounted on a cleat on the scalpel, wherein a guard is moved rearwardly on the scalpel to expose the blade, wherein a blade stripper is moved forwardly to engage the rear portion of the blade and deflect the rear portion of the blade away from the cleat on the scalpel, and wherein the guard is then moved forwardly on the scalpel to strip the blade off the cleat and thus remove the blade from the scalpel.

Accordingly, and in this combination, it is the primary object of the present invention to provide a manually-releasable lock on the blade stripper to prevent an accidental or inadvertent stripping of the blade off the scalpel.

In a preferred embodiment, the lock includes a locking stud carried by a depressible top button on the scalpel, and a hook on the blade stripper receiving the locking stud.

The guard and the blade stripper move longitudinally of the scalpel, respectively, and the locking stud is disposed transversely to the respective longitudinal movement of the guard and blade stripper. Preferably, the depressible top button is part of a leaf spring on the main body portion (or handle) of the scalpel, such that the top button is pivotably disposed.

The depressible top button on the scalpel also carries a transverse detent pin, and the guard has a closed slot within which the detent pin slides. The closed slot has a pair of detent pockets, one at each end thereof for receiving the detent pin, thereby providing a two-position manually-releasable detented position of the guard, and thereby precluding complete removal of the guard off the scalpel. Preferably, the respective detent pin and locking stud on the depressible top button are disposed oppositely of each other.

The blade stripper has a forward edge which is beveled and slides under the rear portion of the blade and cams the blade away from the cleat on the scalpel to position the blade for ejection as the rear portion of the blade rides up the incline on the beveled forward edge of the blade stripper. Preferably, the forward edge of the blade stripper comprises a compound beveled forward edge.

Viewed in another aspect, the present invention constitutes, in combination, a guarded surgical scalpel having a blade stripper, and a lock on the blade stripper to prevent inadvertent or accidental movement of the blade stripper during normal use or handling of the guarded surgical scalpel.

Viewed in yet another aspect, the present invention provides an improved method of using a blade stripper in a guarded surgical scalpel, including the steps of providing a first element carried by the scalpel and a second element carried by the blade stripper and engaging the first element on the scalpel, thereby precluding the inadvertent or accidental movement of the blade stripper during use or handling of the guarded surgical scalpel. The guard is retracted on the scalpel, and the first element on the scalpel is moved away from the second element on the blade stripper, thereby clearing the second element on the blade stripper from the first element on the scalpel. The blade stripper is advanced forwardly of the scalpel to engage the rearward portion of the blade and position it for subsequent engagement by the guard. The guard is then advanced forwardly of the scalpel to engage the positioned rearward portion of the blade and eject the blade from the scalpel.

In a preferred embodiment, the first element on the scalpel comprises a locking stud on a depressible top button formed on a leaf spring portion of the scalpel, and the second element on the blade stripper comprises a hook on the blade stripper receiving the locking stud on the depressible top button.

These and other objects of the present invention will become apparent from a reading of the following specification taken in conjunction with the enclosed drawings.

### BRIEF DESCRIPTION OF THE DRAWINGS

FIG. 1 is a left side elevation of the improved guarded surgical scalpel and combination blade stripper of the present invention. The guard has been extended (advanced forwardly of the scalpel) to enclose the blade.

FIG. 2 is the opposite view of the scalpel of FIG. 1, constituting a right side elevational view. As shown, the detent pin is in one of its cooperating recesses at the respective end of a closed slot formed on the guard. The guard is thereby precluded from completely coming off of the scalpel.

FIG. 3 is a further left side elevation, corresponding substantially to FIG. 1, but having the guard retracted to expose the blade on the scalpel.

FIG. 4 is a portion of FIG. 3, drawn to an enlarged scale, and showing the locking stud on the depressible top button of the scalpel being received in a hook formed on the blade stripper slidably guided for longitudinal movement within the scalpel.

FIG. 5 is a portion of FIG. 4, drawn to a still further enlarged scale, and more clearly showing the locking stud received in the hook.

FIG. 6 is a cross-sectional view, taken along the lines 6-6 of FIG. 4 and showing, respectively, the locking stud and the oppositely-disposed detent pin on the depressible top button of the scalpel.

FIG. 7 corresponds substantially to FIG. 4, but shows the top button being depressed to clear its locking stud from the hook on the blade stripper.

FIG. 8 is a portion thereof, drawn to an enlarged scale, corresponding substantially to FIG. 5, but showing the locking stud completely clear of the hook on the blade stripper.

FIG. 9 is a pictorial view of the scalpel, showing how the thumb on one hand (in this case, the user's right hand) is depressing the top button on the scalpel, and further showing how the thumb (or forefinger) of the user's left hand thereafter slides the blade stripper forwardly of the scalpel.

FIG. 10 is an exploded perspective of the scalpel handle, the guard, the blade stripper and the blade on the cleat forwardly of the scalpel.

FIGS. 11-14 are sequence views, illustrating the operation of the blade stripper (in its unlocked position) and the guard to eject the blade from the scalpel.

FIG. 11 is an elevational view, showing the (unlocked) blade stripper slid forwardly of the scalpel.

FIG. 12 is a further elevational view, corresponding substantially to FIG. 11, but showing the forward, edge of the blade stripper engaging the rear portion of the blade.

FIG. 13 is a top plan view thereof, showing how the forward edge of the blade stripper lifts the rearward portion of the blade and cams it laterally away from the cleat on the scalpel.

FIG. 14 is a further top plan view, corresponding substantially to FIG. 13, but showing the guard moved forwardly on the scalpel to engage the laterally lifted-off rearward portion of the blade to eject the blade (“pop” it off) completely away from the scalpel.

FIG. 15 is a flow chart showing, schematically, the steps in the improved method of the present invention.

### GENERAL DESCRIPTION OF THE PREFERRED EMBODIMENTS

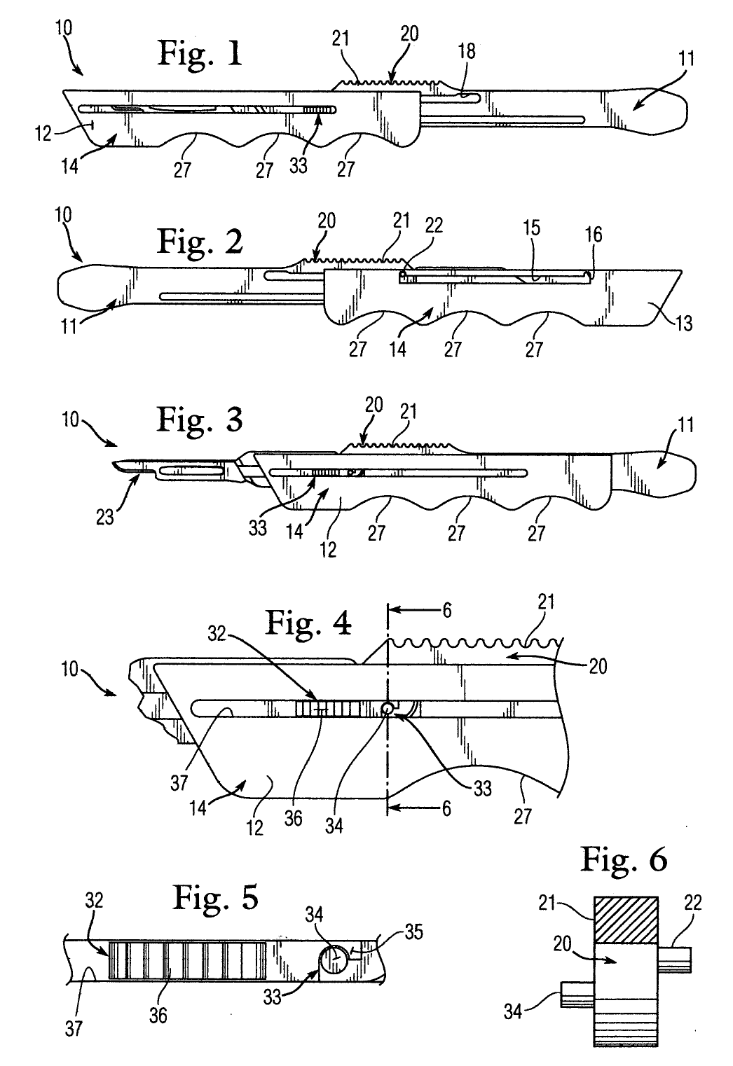

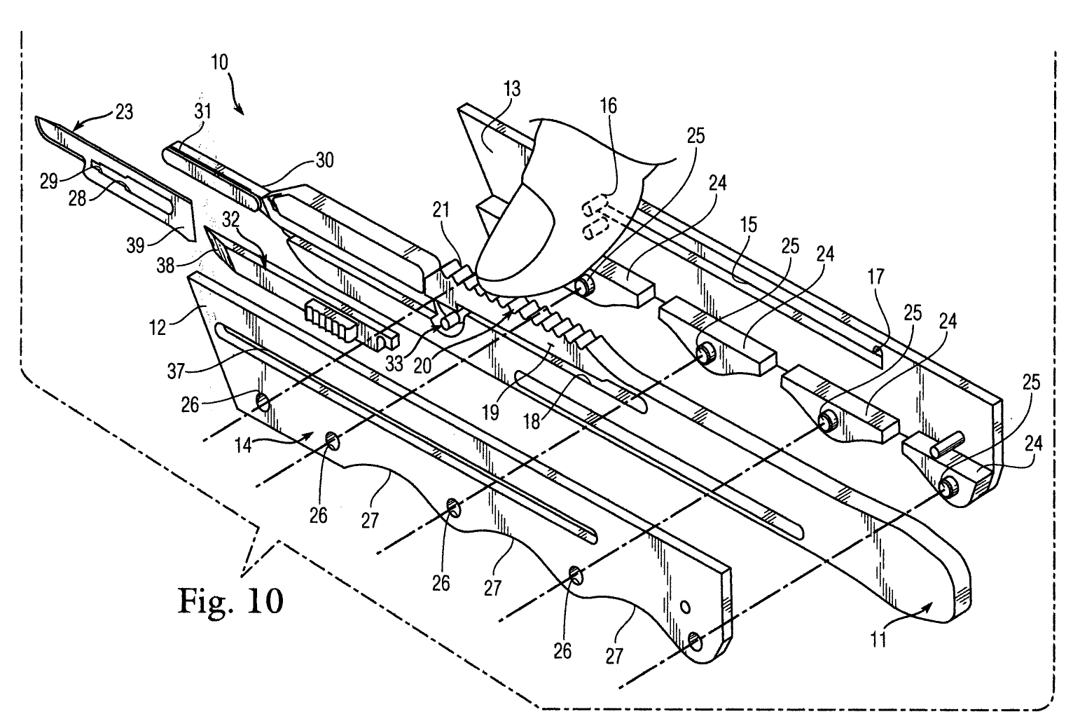

With reference to FIGS. 1-3 and 10, the guarded surgical scalpel 10 includes a main body portion (or “handle”) 11 enclosed between (and carrying) a pair of plates 12 and 13, respectively, the latter being shown more clearly in FIG. 10. The plates 12 and 13 form a guard 14 slidably mounted on the scalpel 10. Plate 13 of the guard 14 has a closed longitudinal slot 15 provided with detent pockets 16 and 17, respectively.

The main body portion 11 of the scalpel 10 (see FIG. 10) has a slot 18 formed therein to provide a leaf spring 19, thereby forming a depressible top button 20 which is thus pivotably disposed. The top button 20 has serrations or grooves 21 to facilitate its manual manipulation. The depressible (pivotable) top button 20 carries a detent pin 22 which rides in the closed longitudinal slot 15 on the guard 14. As shown in FIG. 12, the detent pin 22 is received in the detent pocket 17 when the guard 14 is in its retracted position on the scalpel 10.

While trapping the main body portion or handle 11 of the scalpel 10 between the respective plates 12 and 13 (forming the guard 14), the guard 14 is nevertheless slidably mounted on the handle 11 for alternate advancement and retraction thereon, as defined by the closed slot 15, for alternatively enclosing and exposing a blade 23, respectively.

With reference again to FIG. 10, plate 13 of the guard 14 has a plurality of spaced-apart bosses 24, each of which is provided with a projecting stud 25. During assembly of the overall scalpel 10, these studs 25 are press-filled (an interference fit) within corresponding spaced-apart holes 26 formed in the plate 12 of the guard 14. It will be appreciated, of course, that the dimensions and tolerances of the parts, while retaining the structural integrity of the scalpel 10, nevertheless accommodate the unrestricted relative sliding motion between the guard 14 and scalpel 10. The guard 14, is also provided with finger indents 27 for convenient use of the scalpel 10.

The blade 23 (see FIG. 10) has a slotted opening 28 having a narrowed forwardly-extending portion 29; and a cleat 30 formed on the handle 11 (and extending forwardly thereof) has a pair of slits, one of which is shown at 31 in FIG. 10.

As is well known, the cleat 30 is slipped into the slotted opening 28 in the blade 23 (or vice-versa) and the cleat 30 is slid forwardly so that the slits 31 on the cleat 30 engage within the narrowed forwardly-extending portion 29 of the slotted opening 28 in the blade 23, thereby mounting the blade 23 on the scalpel 10 with substantially a “snap” action.

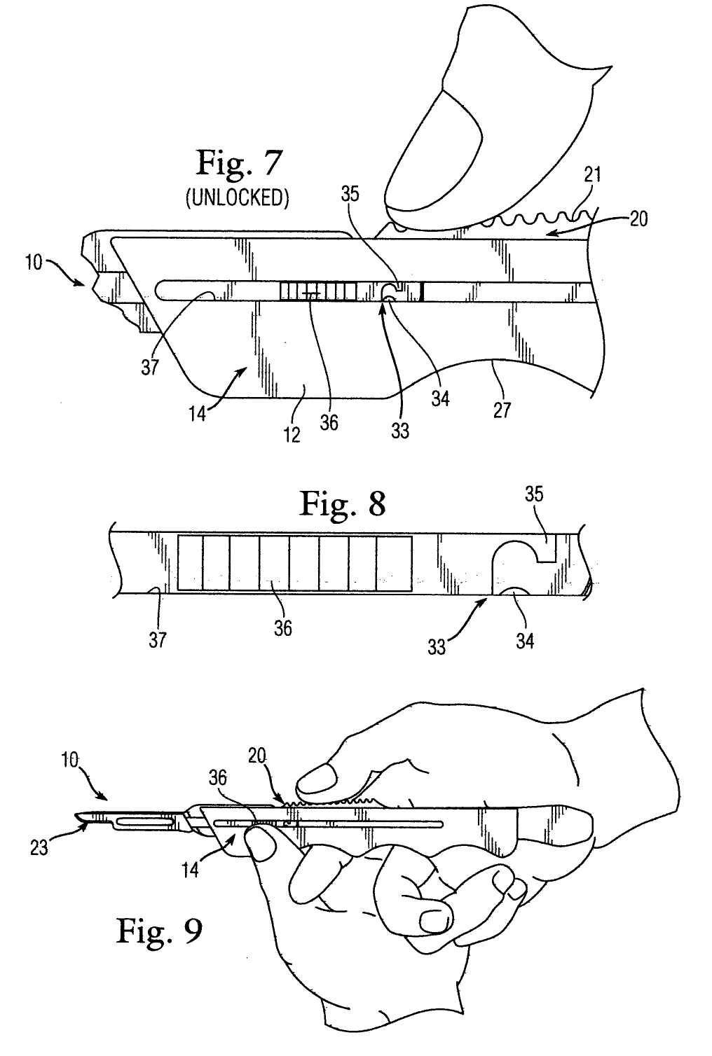

With reference again to FIGS. 1-3 and 10, and with further reference to FIGS. 4-8, a slidable blade stripper 32 is carried on the scalpel 10; and fulfilling the objects of the present invention, a blade stripper lock 33 is provided.

The blade stripper lock 33 of the present invention includes a locking stud 34 carried by the depressible top button 20 and extending laterally therefrom. This locking stud 34 is received within a hook 35 formed on the rearward portion of the blade stripper 32 (see FIGS. 4 and 5) thereby locking the blade stripper 32 against movement on the scalpel 10.

When the top button 20 is pivotably depressed (as shown in FIG. 7) the locking stud 34 clears the hook 35 on the blade stripper 32, thereby allowing the blade stripper 32 to be slid forwardly of the scalpel 10. The blade stripper 32 has a side button 36 slidably guided within a longitudinal slot 37 on plate 12 of the guard 14.

With reference to FIG. 9, the slidable side button 36 of the blade stripper lock 33 and its release by depressing the top button 20, requires a two-handed operation, thereby precluding an inadvertent or accidental engagement of the blade stripper 32—and consequent ejection of the blade 23—during use or handling of the scalpel 10 and, especially, during a surgical procedure in an operating room (or “O.R.”).

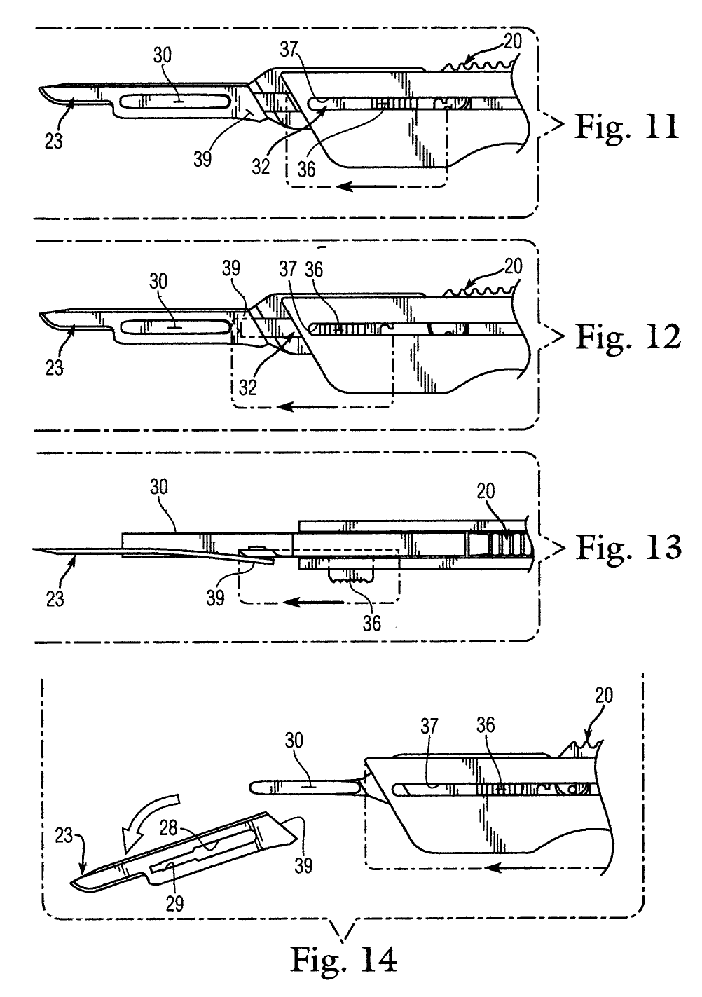

With reference to FIGS. 11-14, the blade stripper 32 has a forward edge formed with a compound beveled edge 38, see FIG. 10, thereby facilitating the insertion of the blade stripper 32 between the blade 23 and the cleat 30 on the scalpel 10 (as shown more clearly in FIG. 13). As a result, the rearward portion 39 of the blade 23 is cammed or lifted away, laterally, from the cleat 30. This positions or aligns the rearward portion 39 of the blade 23 for engagement by the guard 14 (see FIG. 14) so that the blade 23 is ejected (or popped-off) of the scalpel 10.

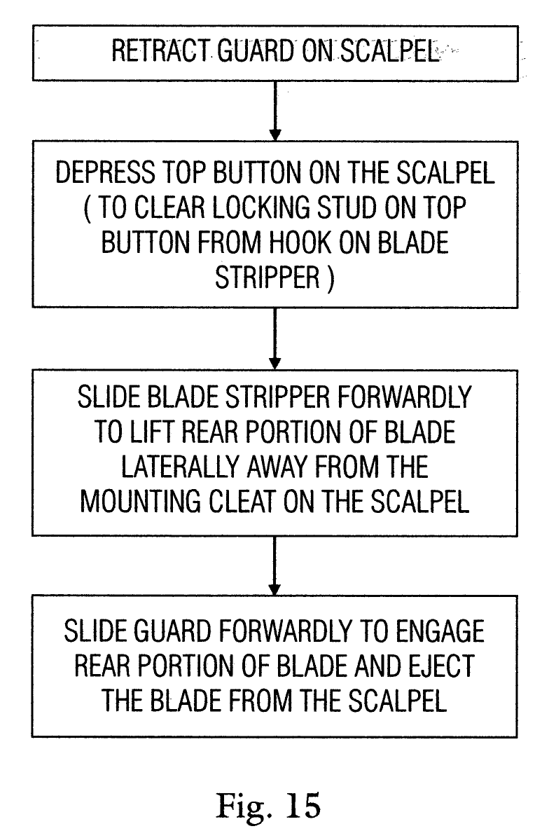

With reference to FIG. 15, the method steps of the present invention are as follows: First, the guard 14 is slidably retracted (rearwardly) of the scalpel 10, thus exposing the (used) blade 23. Second, the top button 20 on the scalpel 10 is pivotably depressed inwardly of the scalpel 10—the top button 20 is formed on the leaf spring 19—so that the locking stud 34 on the top button 20 is disengaged from the hook 35 on the blade stripper 32. The blade stripper 32 is now unlocked. Third, the blade stripper 32 is slid forwardly of the scalpel 10 by means of the side button 36 riding in the longitudinal slot 37 in the side plate 12 of the guard 14. The knife edge on the blade stripper 32—having a compound beveled edge—thus engages and lifts off or cams the rearward portion 39 of the blade 23 laterally away from the cleat 30 on the scalpel 10. This positions the blade for engagement by the guard 14. Finally, the guard 14 is slid forwardly to push the blade 23 off the cleat 30 and eject the blade 23 from the scalpel 10 (as previously explained).

This whole operation (see FIG. 9) may be effected easily and conveniently, in a two-handed operation, without any risk of an inadvertent or accidental ejection of the blade 23, especially during a surgical procedure.

Premature ejection of the blade 23 is thereby prevented by the blade stripper lock 33 of the present invention.

Obviously, many modifications may be made without departing from the basic spirit of the present invention. Accordingly, it will be appreciated by those skilled in the art that within the scope of the appended claims, the invention may be practiced other than has been specifically described herein.

1. In a guarded surgical scalpel, wherein a blade is mounted on a cleat on the scalpel, wherein a guard is moved rearwardly on the scalpel to expose the blade, wherein a blade stripper is moved forwardly to engage the rear portion of the blade and deflect the rear portion of the blade away from the cleat on the scalpel, and wherein the guard is then moved forwardly on the scalpel to strip the blade off the cleat and thus remove the blade from the scalpel, the improvement comprising a manually-releasable lock on the blade stripper to prevent an accidental or inadvertent stripping of the blade off the scalpel.

2. The improvement of claim 1, wherein the scalpel includes a depressible top button, and wherein the lock comprises a locking stud carried by the depressible top button, and a hook on the blade stripper receiving the locking stud.

3. The improvement of claim 2, wherein the guard and the blade stripper move longitudinally of the scalpel, respectively, and wherein the locking stud is disposed transversely to the respective longitudinal movement of the guard and blade stripper.

4. The improvement of claim 2, wherein the depressible top button is part of a leaf spring that is pivotably disposed within the scalpel.

5. The improvement of claim 2, wherein the depressible top button also carries a transverse detent pin, wherein the guard has a closed slot within which the detent pin slides, and wherein the closed slot has a pair of detent pockets, one at each end thereof for receiving the detent pin, thereby providing a two-position manually-releasable detented position of the guard, and thereby precluding complete removal of the guard off the scalpel.

6. The improvement of claim 5, wherein the respective detent pin and locking stud on the depressible top button are disposed oppositely of each other.

7. The improvement of claim 1, wherein the blade stripper has a forward edge which is beveled and includes an incline, and wherein the beveled forward edge slides under the rear portion of the blade and cams the blade away from the cleat on the scalpel to eject the blade as the rear portion of the blade rides up the incline on the beveled forward edge of the blade stripper.

8. The improvement of claim 6, wherein the forward edge of the blade stripper comprises a compound beveled forward edge.

9. In a guarded surgical scalpel, wherein a blade is mounted on a cleat on the main body portion of the scalpel, wherein a guard is moved rearwardly on the main body portion of the scalpel to expose the blade, wherein a blade stripper is moved forwardly to engage the rear portion of the blade and deflect the rear portion of the blade away from the cleat on the scalpel, and wherein the guard is then moved forwardly on the scalpel to strip the blade off the cleat and thus remove the blade from the scalpel, the guard and blade stripper moving longitudinally of the scalpel, the improvement comprising a manually-releasable blade stripper lock to prevent an accidental or inadvertent stripping of the blade off the scalpel, the blade stripper lock including a depressible top button, the depressible top button carrying a locking stud disposed transversely of the longitudinal sliding movement of the guard, the depressible top button being formed on a leaf spring that is pivotably disposed within the main body portion of the scalpel, a hook on the blade stripper receiving the locking stud, wherein the depressible top button also carries a transverse detent pin, wherein the guard has a closed slot within which the detent pin slides, and wherein the closed slot has a pair of detent pockets, one at each end thereof for receiving the detent pin, thereby providing a two-position manually-releasable detented position of the guard, and thereby precluding complete removal of the guard off the scalpel, wherein the respective detent pin and locking stud on the depressible top button are disposed oppositely of each other, and wherein the blade stripper has a forward edge which is beveled and includes an incline, and wherein the beveled forward edge slides under the rear portion of the blade and cams the blade away from the cleat on the scalpel to position the blade for ejection as the rear portion of the blade rides up the incline on the beveled forward edge of the blade stripper, and thereafter the guard is moved forward to eject the blade.

10. In combination, a guarded surgical scalpel having a blade stripper, and a blade stripper lock to prevent inadvertent or accidental movement of the blade stripper during normal use or handling of the guarded surgical scalpel.

11. The combination of claim 10, wherein the guarded surgical scalpel includes a handle and further includes a guard adapted for longitudinal sliding movement thereon, and wherein the blade stripper lock comprises a button on the handle of the scalpel, the button carrying a locking stud disposed transversely of the longitudinal sliding movement of the guard, and a hook on blade stripper to receive the locking stud.

12. The combination of claim 11, wherein the button on the scalpel comprises a depressible top button.

13. The combination of claim 12, wherein the depressible top button is carried by a leaf spring on the handle of the scalpel.

14. In a blade stripper for a guarded surgical scalpel, the improvement wherein the blade stripper has a forward edge engaging the rearward portion of a blade mounted on a cleat on the scalpel, and wherein the forward edge of the blade stripper is formed with a compound beveled edge.

15. The improvement of claim 14, further including a lock for the blade stripper, thereby precluding inadvertent or accidental movement of the blade stripper during use or handling of the guarded surgical scalpel.

16. The method of using a blade stripper in a guarded surgical scalpel, wherein the scalpel carries a blade mounted forwardly thereon, and wherein the scalpel is provided with a guard and is further provided with a blade stripper, comprising the steps of providing a first element carried by the scalpel and a second element carried by the blade stripper and engaging the first element on the scalpel, thereby precluding inadvertent or accidental movement of the blade stripper during use or handling of the guarded surgical scalpel, retracting the guard on the scalpel, moving the first element on the scalpel away from the second element on the blade stripper, thereby clearing the second element on the blade stripper from the first element on the scalpel, advancing the blade stripper forwardly of the scalpel to engage the rearward portion of the blade and position the rearward portion of the blade for subsequent engagement by the guard, and advancing the guard forwardly of the scalpel to engage the positioned rearward portion of the blade and eject it from the scalpel.

17. The method of claim 16, wherein the first element on the scalpel comprises a locking stud on a depressible top button formed on a leaf spring portion of the scalpel, and wherein the second element on the blade stripper comprises a hook on the blade stripper receiving the locking stud on the depressible top button.
